(19) United States
(12) Patent Application Publication
(10) Pub. No.: US 2026/0225136 A1
(43) Pub. Date: Aug. 6, 2026

(54) METHODS OF CLEANING CHROMATOGRAPHY MATRICES

(71) Applicant: ELI LILLY AND COMPANY, Indianapolis, IN (US)

(72) Inventors: Noah TAN SHIZHAO, Rochestown (IE); Jean LORDAN, Glounthaune (IE)

(21) Appl. No.: 19/141,230

(22) PCT Filed: Dec. 19, 2023
(86) PCT No.: PCT/US2023/084938

§ 371 Date: Jun. 19, 2025

Related U.S. Application Data
(60) Provisional application No. 63/476,080, filed on Dec. 19, 2022.

(51) Int. Cl. B08B 3/08; C07K 14/605; C11D 3/04; C11D 3/20; C11D 3/30

(52) U.S. Cl. CPC B08B 3/08; C07K 14/605; C11D 3/042; C11D 3/046; C11D 3/2079; C11D 3/30

(57) ABSTRACT

Provided herein are improved methods of cleaning a protein A chromatography matrix that has previously been used to purify an Fc-containing protein. The methods generally involve washing the protein A chromatography matrix with a series of three solutions, the first containing an acid, the second containing Tris, and the third containing NaOH (e.g., 0.001-0.075 M NaOH).

### BACKGROUND

Production of recombinant Fc-containing proteins for therapeutic use typically involves expression of the proteins in mammalian cells and subsequent purification of these proteins from host cell contaminants. Protein A affinity chromatography is commonly used as part of the purification process for Fc-containing proteins, because of the ability of protein A to selectively bind to the Fc region of the Fc-containing proteins. To reduce the production costs associated with protein A affinity chromatography, protein A chromatography matrices are typically cleaned and reused multiple times. However, the cleaning of protein A matrices is challenging because the chemicals that are effective at cleaning protein A matrices can also damage the matrices, thereby reducing their useful life.

Accordingly, there is a need for improved methods of cleaning protein A chromatography matrices that prolong the useful life of the matrices.

### SUMMARY

The present disclosure provides improved methods for cleaning a protein A chromatography matrix. The methods generally involve washing the protein A chromatography matrix with a series of three solutions, the first containing an acid, the second containing Tris, and the third containing NaOH (e.g., 0.001-0.075 M NaOH). The methods disclosed herein are particularly advantageous in that they can significantly increase the useful life of a protein A chromatography matrix, and thereby reduce the cost of purifying Fc-containing proteins.

In an aspect, provided herein is a method for cleaning a protein A chromatography matrix that has previously been used to purify dulaglutide, the method comprising sequentially washing a chromatography column comprising the protein A chromatography matrix with: a) a first solution comprising one or more acid; b) a second solution comprising Tris; and c) a third solution comprising 0.001-0.075 M NaOH.

In an embodiment, the first solution comprises acetic acid. In an embodiment, the first solution comprises phosphoric acid. In an embodiment, the first solution comprises acetic acid and phosphoric acid. In an embodiment, the first solution comprises 0.5-5% acetic acid. In an embodiment, the first solution comprises 0.5-1.5% acetic acid. In an embodiment, the first solution comprises about 1% acetic acid. In an embodiment, the first solution comprises 0.5-5% phosphoric acid. In an embodiment, the first solution comprises 0.5-1.5% phosphoric acid. In an embodiment, the first solution comprises about 1% phosphoric acid. In an embodiment, the first solution comprises about 1% acetic acid and about 1% phosphoric acid.

In an embodiment, the second solution has a pH of 7-9. In an embodiment, the second solution has a pH of about 8. In an embodiment, the second solution comprises 10-100 mM Tris. In an embodiment, the second solution comprises 25-75 mM Tris. In an embodiment, the second solution comprises about 50 mM Tris.

In an embodiment, the third solution comprises 0.005-0.05 M NaOH. In an embodiment, the third solution comprises 0.009-0.015 M NaOH. In an embodiment, the third solution comprises about 0.01 M NaOH.

In an embodiment, the protein A chromatography matrix is washed with 1-10 column volumes of the first solution. In an embodiment, the protein A chromatography matrix is washed with 2-3 column volumes of the first solution. In an embodiment, the protein A chromatography matrix is washed with 2-10 column volumes of the second solution. In an embodiment, the protein A chromatography matrix is washed with 1-2 column volumes of the second solution. In an embodiment, the protein A chromatography matrix is washed with 2-10 column volumes of the third solution. In an embodiment, the protein A chromatography matrix is washed with 2-3 column volumes of the third solution.

In an embodiment, the protein A chromatography matrix is washed with the first solution at a flow rate of about 270 cm/hr. In an embodiment, the protein A chromatography matrix is washed with the second solution at a flow rate of about 270 cm/hr. In an embodiment, the protein A chromatography matrix is washed with the third solution at a flow rate of about 270 cm/hr.

In an embodiment, the chromatography column has a diameter of 75-150 cm. In an embodiment, the chromatography column has a diameter of about 100 cm. In an embodiment, the chromatography column has a diameter of about 140 cm.

In an embodiment, the protein A chromatography matrix has an average particle size of 80-90 μm. In an embodiment, the protein A chromatography matrix has an average particle size of about 85 μm.

In an embodiment, the protein A chromatography matrix a comprises a protein A ligand that has increased stability under alkali conditions relative to wild type Staphylococcus aureus protein A.

In an embodiment, the first solution comprises about 1% acetic acid and about 1% phosphoric acid; the second solution comprises about 50 mM Tris at a pH of about 8; and the third solution comprises about 0.01 M NaOH.

In an embodiment, the protein A chromatography matrix is washed with: 2-3 column volumes of the first solution; b) 1-2 column volumes of the second solution; and c) 2-3 column volumes of the third solution. In an embodiment, the protein A chromatography matrix is washed with the first, second, and third solutions at a flow rate of about 270 cm/hr.

In an embodiment, the method further comprises washing the protein A chromatography matrix with about 2 column volumes of the second solution after the protein A chromatography matrix is washed with the third solution. In an embodiment, the method further comprises washing the protein A chromatography matrix with a fourth solution comprising about 0.1 M NaOH.

In an embodiment, the protein A chromatography matrix is washed with about 2 column volumes of the fourth solution. In an embodiment, the protein A chromatography matrix is washed with the fourth solution at a flow rate of 100-140 cm/hr. In an embodiment, the protein A chromatography matrix is washed with the fourth solution at a flow rate of about 130 cm/hr. In an embodiment, the protein A chromatography matrix is washed with the fourth solution at a flow rate of 100-110 cm/hr. In an embodiment, the protein A chromatography matrix is washed with the fourth solution for about 25 minutes.

In an embodiment, the protein A chromatography matrix is washed with the first solution, second solution, and/or third solution in an upflow or downflow direction.

In an embodiment, the protein A chromatography matrix has less than 1% carryover from the previous use after the protein A chromatography matrix is cleaned. In an embodiment, the protein A chromatography matrix has less than 0.1% carryover from the previous use after the protein A chromatography matrix is cleaned.

In an embodiment, the protein A chromatography matrix is used for 200-500 cycles of dulaglutide purification. In an embodiment, the protein A chromatography matrix is used for 300-400 cycles of dulaglutide purification. In an embodiment, the protein A chromatography matrix is used for about 304 cycles of dulaglutide purification.

Additional embodiments of the present disclosure are described below:
1. A method for cleaning a protein A chromatography matrix, the method comprising sequentially washing a chromatography column comprising the protein A chromatography matrix with:
   a) a first solution comprising one or more acid;
   b) a second solution comprising Tris; and
   c) a third solution comprising 0.001-0.075 M NaOH.
2. The method of embodiment 1, wherein the first solution comprises acetic acid.
3. The method of embodiment 1 or 2, wherein the first solution comprises phosphoric acid.
4 The method of any one of embodiments 1-3, wherein the first solution comprises acetic acid and phosphoric acid.
5. The method of any one of embodiments 1-4, wherein the first solution comprises 0.5%-5% acetic acid.
6. The method of any one of embodiments 1-5, wherein the first solution comprises 0.5%-1.5% acetic acid.
7. The method of any one of embodiments 1-6, wherein the first solution comprises about 1% acetic acid.
8. The method of any one of embodiments 1-7, wherein the first solution comprises 0.5%-5% phosphoric acid.
9. The method of any one of embodiments 1-8, wherein the first solution comprises 0.5%-1.5% phosphoric acid.
10. The method of any one of embodiments 1-9, wherein the first solution comprises about 1% phosphoric acid.
11. The method of any one of embodiments 1-10, wherein the first solution comprises about 1% acetic acid and about 1% phosphoric acid.
12. The method of any one of embodiments 1-11, wherein the second solution has a pH of 7-9.
13. The method of any one of embodiments 1-12, wherein the second solution has a pH of about 8.
14. The method of any one of embodiments 1-13, wherein the second solution comprises 10-100 mM Tris.
15. The method of any one of embodiments 1-14, wherein the second solution comprises 25-75 mM Tris.
16. The method of any one of embodiments 1-15, wherein the second solution comprises about 50 mM Tris.
17. The method of any one of embodiments 1-16, wherein the third solution comprises 0.005-0.05 M NaOH.
18. The method of any one of embodiments 1-17, wherein the third solution comprises 0.009-0.015 M NaOH.
19. The method of any one of embodiments 1-18, wherein the third solution comprises about 0.01 M NaOH.
20. The method of any one of embodiments 1-19, wherein the protein A chromatography matrix is washed with 1-10 column volumes of the first solution.
21. The method of any one of embodiments 1-20, wherein the protein A chromatography matrix is washed with 2-3 column volumes of the first solution.
22. The method of any one of embodiments 1-21, wherein the protein A chromatography matrix is washed with 2-10 column volumes of the second solution.
23. The method of any one of embodiments 1-22, wherein the protein A chromatography matrix is washed with 1-2 column volumes of the second solution.
24. The method of any one of embodiments 1-23, wherein the protein A chromatography matrix is washed with 2-10 column volumes of the third solution.
25. The method of any one of embodiments 1-24, wherein the protein A chromatography matrix is washed with 2-3 column volumes of the third solution.
26. The method of any one of embodiments 1-25, wherein the protein A chromatography matrix is washed with the first solution at a flow rate of about 270 cm/hr.
27. The method of any one of embodiments 1-26, wherein the protein A chromatography matrix is washed with the second solution at a flow rate of about 270 cm/hr.
28. The method of any one of embodiments 1-27, wherein the protein A chromatography matrix is washed with the third solution at a flow rate of about 270 cm/hr.
29. The method of any one of embodiments 1-28, wherein the chromatography column has a diameter of 75-150 cm.
30. The method of any one of embodiments 1-29, wherein the chromatography column has a diameter of about 100 cm.
31. The method of any one of embodiments 1-29, wherein the chromatography column has a diameter of about 140 cm.
32. The method of any one of embodiments 1-31, wherein the Protein A chromatography matrix has an average particle size of 80-90 μm.
33. The method of any one of embodiments 1-32, wherein the Protein A chromatography matrix has an average particle size of about 85 μm.
34. The method of any one of embodiments 1-33, wherein the protein A chromatography matrix a comprises a protein A ligand that has increased stability under alkali conditions relative to wild type Staphylococcus aureus protein A.
35. The method of any one of embodiments 1-34, wherein
   a) the first solution comprises about 1% acetic acid and about 1% phosphoric acid;
   b) the second solution comprises about 50 mM Tris at a pH of about 8; and
   c) the third solution comprises about 0.01 M NaOH.
36. The method of embodiment 35, wherein the protein A chromatography matrix is washed with:
   a) 2-3 column volumes of the first solution;
   b) 1-2 column volumes of the second solution; and
   c) 2-3 column volumes of the third solution.
37. The method of embodiment 35 or 36, wherein the protein A chromatography matrix is washed with the first, second, and third solutions at a flow rate of about 270 cm/hr.
38. The method of any one of embodiments 1-37, further comprising washing the protein A chromatography matrix with about 2 column volumes of the second solution after the protein A chromatography matrix is washed with the third solution.
39. The method of any one of embodiments 1-38, further comprising washing the protein A chromatography matrix with a fourth solution comprising about 0.1 M NaOH.
40. The method of embodiment 39, wherein the protein A chromatography matrix is washed with about 2 column volumes of the fourth solution.
41. The method of embodiment 39 or 40, wherein the protein A chromatography matrix is washed with the fourth solution at a flow rate of 100-110 cm/hr.
42. The method of embodiment 39 or 40, wherein the protein A chromatography matrix is washed with the fourth solution at a flow rate of about 130 cm/hr.
43. The method of any one of embodiments 39-42, wherein the protein A chromatography matrix is washed with the fourth solution for about 25 minutes.
44. The method of any one of embodiments 1-43, wherein the protein A chromatography matrix is washed with the first solution, second solution, and/or third solution in an upflow or downflow direction.
45. The method of any one of embodiments 1-44, wherein the protein A chromatography matrix has previously been used to purify an Fc-containing protein.
46. The method of embodiment 45, wherein the Fc-containing protein is an antibody.
47. The method of embodiment 45, wherein the Fc-containing protein is not an antibody.
48. The method of embodiment 45, wherein the Fc-containing protein comprises a glucagon-like peptide 1 (GLP-1) analog comprising one or more modifications compared to a wild type GLP-1 amino acid sequence (SEQ ID NO: 1).
49. The method of embodiment 45 or 48, wherein the Fc-containing protein comprises a GLP-1 analog comprising the amino acid sequence of SEQ ID NO: 2.
50. The method of any one of embodiments 45, 48, or 49, wherein the Fc-containing protein comprises a peptide linker.
51. The method of embodiment 50, wherein the peptide linker comprises 1 to 10 G₄S units (SEQ ID NO: 6).
52. The method of any one of embodiments 44 or 47-51, wherein the Fc-containing protein comprises:
   a) a GLP-1 analog comprising the amino acid sequence of SEQ ID NO: 2;
   b) a peptide linker comprising 1 to 10 G₄S units (SEQ ID NO: 6); and
   c) an Fc portion of an immunoglobulin.
53. The method of embodiment 52, wherein the N-terminal residue of the peptide linker is directly fused to the C-terminal residue of the GLP-1 analog, and the C-terminal residue of the peptide linker is directly fused to the N-terminal residue of the Fc portion.
54. The method of any one of embodiments 44 or 47-53, wherein the Fc-containing protein comprises the amino acid sequence of SEQ ID NO: 5.
55. The method of any one of embodiments 44 or 47-54, wherein the Fc-containing protein is a homodimer comprising two identical amino acid chains each comprising the amino acid sequence of SEQ ID NO: 5.
56. The method of any one of embodiments 44 or 47-55, wherein the Fc-containing protein is dulaglutide.
57. The method of any one of embodiments 44-56, wherein the protein A chromatography matrix has less than 1% carryover from the previous use.
58. The method of any one of embodiments 44-57, wherein the protein A chromatography matrix has less than 0.1% carryover from the previous use.
59. The method of any one of embodiments 1-58, wherein the protein A chromatography matrix is used for 200-500 cycles.
60. The method of any one of embodiments 1-59, wherein the protein A chromatography matrix is used for more than 250-400 cycles of dulaglutide purification.
61. The method of any one of embodiments 1-60, wherein the protein A chromatography matrix is used for more than 300-400 cycles of dulaglutide purification.
62. The method of any one of embodiments 1-61, wherein the protein A chromatography matrix is used for about 304 cycles.
63. The method of any one of the preceding embodiments, wherein the method does not include a static hold of the first solution.
64. The method of any one of the preceding embodiments, wherein the method does not include a static hold of the second solution.
65. The method of any one of the preceding embodiments, wherein the method does not include a static hold of the third solution.

### BRIEF DESCRIPTION OF DRAWINGS

FIG. 1A-1B are plots showing the neutralized low pH viral inactivation (LpHVI) intermediate pH (FIG. 1A) and conductivity (FIG. 1B) for each of the 304 cycles over the course of the study.

FIG. 2 is a graph that depicts the % yield and dynamic binding capacity (DBC) response for the protein A chromatography matrix throughout the 304 cycles of the study. The % yield for high loads (18 g/L) is shown in green and low loads (10 g/L) is shown in orange. The horizontal black dashed lines indicate minimum and maximum process validation acceptance criteria (PVAC) of 70% and 105%, respectively.

FIG. 3 is a graph that depicts Height Equivalent to Theoretical Plate (HETP) and Asymmetry assessments for the packed protein A chromatography matrix bed as metrics to measure column pack quality. The black dotted line indicates the minimum acceptance criterion of at least 1100 plates/m for HETP and the red dotted lines indicate the minimum and maximum acceptance criteria asymmetry values of 0.7 and 1.8, respectively.

FIG. 4 is a plot that depicts the percent carryover for after mock elution of the protein A chromatography matrix throughout the 304 cycles of the study.

FIG. 5A-5B are plots that depict a product purity assessment as determined by size exclusion chromatography. FIG. 5A is a plot showing the percentage of dulaglutide monomer. The horizontal dashed red line represents the minimum acceptance criteria of 91.2% monomer applied at low pH viral inactivation unit operation. FIG. 5B is a plot showing the percentage of total aggregates. Data points in yellow represent unincubated low pH viral inactivation samples.

FIG. 6 is a plot showing a product purity assessment as determined by measuring residual host cell proteins (rHCPs) in neutralized low pH viral inactivation samples. The horizontal red dashed line represents the maximum acceptance criteria of 482 ppm applied at low pH viral inactivation unit operation.

FIG. 7 is a plot showing a product purity assessment as determined by measuring residual protein A (rProA) leached from the column in neutralized low pH viral inactivation samples. The horizontal red dashed line represents the maximum acceptance criteria of 523 ppm applied at low pH viral inactivation unit operation.

FIG. 8 is a plot showing a product purity assessment as determined by measuring residual DNA in neutralized low pH viral inactivation samples. The horizontal red dashed line represents the maximum acceptance criteria of 500 ppb applied at AEX unit operation.

FIG. 9 is a plot showing a product purity assessment as determined by measuring residual Triton X-100 surfactant in neutralized low pH viral inactivation samples. The horizontal red dashed line represents the maximum acceptance criteria of 518,000 ppm.

FIG. 10 is a graph showing a comparison of percent yield in a previous protein A chromatography matrix lifetime study compared to the present lifetime study. Outliers are represented by grey dots.

### DETAILED DESCRIPTION

The present disclosure provides improved methods for cleaning a protein A chromatography matrix. The methods generally involve washing the protein A chromatography matrix with a series of three solutions, the first containing an acid, the second containing Tris, and the third containing NaOH (e.g., 0.001-0.075 M NaOH). The methods disclosed herein are particularly advantageous in that they can significantly increase the useful life of a protein A chromatography matrix, and thereby reduce the cost of purifying Fc-containing proteins.

### I. Definitions

As used herein the term “cleaning” refers to removing residual material (e.g., protein) bound to a chromatography matrix after the matrix has been used for purification of an Fc-containing protein (e.g., dulaglutide). In an embodiment, the cleaning comprises both a regeneration and a sanitization step.

As used herein, the term “upflow” refers to flowing a solution upwards through a chromatography column.

As used herein, the term “downflow” refers to flowing a solution or buffer downwards through a chromatography column.

As used herein, the term “carryover” refers to protein and other impurities that remain bound to a chromatography matrix after cleaning of the protein A chromatography matrix, as measured by a mock elution. In an embodiment, the carryover is calculated as a percent of the total peak area of eluted Fc-containing protein (e.g., dulaglutide) in the prior cycle based on an A280 pathlength of 2 mm as follows: (mock elution peak area (ml*mAU)/prior cycle elution peak area (ml*mAU)×100=% carryover.

As used herein the term “mock elution” refers to an elution procedure that is applied to a chromatography matrix for which a protein was not loaded subsequent to the last cleaning procedure.

As used herein, the term “Fc-containing protein” refers to a protein comprising an Fc region. In an embodiment, the Fc-containing protein comprises a variant Fc region comprising one or more amino acid substitutions, additions, and/or deletions relative to a naturally occurring Fc region. In an embodiment, the Fc-containing protein is an antibody. In an embodiment, the Fc-containing protein is not an antibody.

As used herein, the term “contaminant” refers to any material, particularly a biological macromolecule such as DNA, RNA, or a protein, other than a recombinantly produced Fc-containing protein that is present in a mixture. Contaminants include, without limitation, cellular and viral proteins or nucleic acids, or byproducts thereof, that arise in the production process of an Fc-containing protein. A contaminant also includes any host cell protein (HCP), host cell nucleic acid, or host cell fragment that results from any stage of an Fc-containing protein production process.

The terms “host cell protein,” and “HCP,” are used herein to refer to any unwanted protein that originates from a cell (e.g., a mammalian cell) used to produce an Fc-containing protein.

As used herein, the term “purifying,” “purify,” or “purification” refers to reduction in the amount of a contaminant (e.g., an HCP) in a composition comprising an Fc-containing protein. Purification may or may not result in the complete removal of contaminants from a composition. In certain embodiments, purification refers to at least a 2-fold, 3-fold, 4-fold, 5-fold, 10-fold, 15-fold, 20-fold, 25-fold, 30-fold, 35-fold, 40-fold, 45-fold, or 50-fold reduction in contaminants.

As used herein, the term “antibody” includes full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above.

As used herein, the term “about,” when in reference to a value or parameter herein, includes a variability of ±5% of the value or parameter. For example, when referring to a pH value, “about” refers to a range that includes the value 5% below the referenced value, and the value 5% above the referenced value. Thus, a pH of about 10 refers to a pH that encompasses a pH of 9.5 to a pH of 10.5, inclusive.

### II. Methods of Cleaning Protein A Chromatography Matrices

Cleaning protein A chromatography matrices is challenging because the chemicals that are effective at cleaning protein A matrices can also damage the matrices, thereby reducing their useful life. The methods disclosed herein significantly increase the usable lifetime of a protein A chromatography matrix, for example a protein A chromatography matrix that has been used to purify an Fc-containing protein (e.g., dulaglutide), by minimizing the carryover without damaging the matrix.

In general, a cycle of purification using a protein A chromatography method comprises the following steps in sequential order: preparing a load composition comprising the Fc-containing protein, applying the load composition to the chromatography column comprising the protein A chromatography matrix, washing of the protein A chromatography matrix, eluting the Fc-containing protein, and cleaning the protein A chromatography matrix. The skilled artisan will appreciate that depending on the desired purpose and outcome, a cycle of purification using a protein A chromatography method can comprise additional intermediate steps, and/or additional steps before and/or after the protein A purification process.

In an aspect, provided herein is a method for cleaning a protein A chromatography matrix that has previously been used to purify an Fc-containing protein, the method comprising sequentially washing a chromatography column comprising the protein A chromatography matrix with: a) a first solution comprising one or more acid; b) a second solution comprising Tris; and c) a third solution comprising 0.001-0.075 M NaOH.

In an aspect, provided herein is a method for cleaning a protein A chromatography matrix that has previously been used to purify dulaglutide, the method comprising sequentially washing a chromatography column comprising the protein A chromatography matrix with: a) a first solution comprising one or more acid; b) a second solution comprising Tris; and c) a third solution comprising 0.001-0.075 M NaOH.

The cleaning solutions and methods are described in detail below.

### Cleaning Solutions and Methods

After a Protein A chromatography matrix has been used to purify an Fc-containing protein (e.g., dulaglutide), the Protein A chromatography matrix is cleaned by washing the matrix with a series of solutions to remove residual protein and contaminants from the matrix, without damaging the matrix. In an aspect, after a chromatography column comprising a Protein A chromatography matrix has been used to purify an Fc-containing protein, the protein A chromatography matrix is sequentially washed with a) a first solution comprising one or more acid; b) a second solution comprising Tris; and c) a third solution comprising NaOH (0.001-0.075 M). The methods disclosed herein are particularly advantageous in that they can significantly increase the useful life of a protein A chromatography column matrix, and thereby reduce the cost of purifying Fc-containing proteins.

In an embodiment, the first solution comprises acetic acid. In an embodiment, the first solution comprises phosphoric acid. In an embodiment, the first solution comprises acetic acid and phosphoric acid.

In an embodiment, the first solution comprises 0.5-5% acetic acid. In an embodiment, the first solution comprises 0.5-1.5% acetic acid. In an embodiment, the first solution comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% acetic acid. In an embodiment, the first solution comprises about 1% acetic acid.

In an embodiment, the first solution comprises 0.5-5% phosphoric acid. In an embodiment, the first solution comprises 0.5-1.5% phosphoric acid. In an embodiment, the first solution comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% phosphoric acid. In an embodiment, the first solution comprises about 1% phosphoric acid.

In an embodiment, the first solution comprises 0.5-5% acetic acid and about 1% phosphoric acid. In an embodiment, the first solution comprises 0.5-1.5% acetic acid. In an embodiment, the first solution comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% acetic acid and about 1% phosphoric acid.

In an embodiment, the first solution comprises about 1% phosphoric acid and 0.5-5% phosphoric acid. In an embodiment, the first solution comprises about 1% phosphoric acid and 0.5-1.5% phosphoric acid. In an embodiment, the first solution comprises about 1% phosphoric acid and about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% phosphoric acid.

In an embodiment, the first solution comprises 0.5-5% acetic acid and 0.5-5% phosphoric acid. In an embodiment, the first solution comprises 0.5-1.5% acetic acid and 0.5-1.5% phosphoric acid. In an embodiment, the first solution comprises about 1% acetic acid and about 1% phosphoric acid.

In an embodiment, the second solution has a pH of 7-9. In an embodiment, the second solution has a pH of about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In an embodiment, the second solution has a pH of about 8.

In an embodiment, the second solution comprises 10-100 mM Tris. In an embodiment, the second solution comprises 25-75 mM Tris. In an embodiment, the second solution comprises about 10, about 15, about 20, about 25, about 30, about 35, about 40, about 45, about 50, about 55, about 60, about 65, about 70, about 75, about 80, about 85, about 90, about 95, or about 100 mM Tris. In an embodiment, the second solution comprises about 50 mM Tris.

In an embodiment, the second solution comprises 10-100 mM Tris and has a pH of about 8. In an embodiment, the second solution comprises 25-75 mM Tris and has a pH of about 8. In an embodiment, the second solution comprises about 10, about 15, about 20, about 25, about 30, about 35, about 40, about 45, about 50, about 55, about 60, about 65, about 70, about 75, about 80, about 85, about 90, about 95, or about 100 mM Tris and has a pH of about 8. In an embodiment, the second solution comprises about 50 mM Tris and has a pH of about 8.

In an embodiment, the third solution comprises 0.005-0.05 M NaOH. In an embodiment, the third solution comprises 0.009-0.015 M NaOH. In an embodiment, the third solution comprises about 0.005 M, about 0.006 M, about 0.007 M, about 0.008 M, about 0.009 M, about 0.01 M, about 0.011 M, about 0.012 M, about 0.013 M, about 0.014 M, about 0.015 M, about 0.016 M, about 0.017 M, about 0.018 M, about 0.019 M, about 0.02 M, about 0.021 M, about 0.022 M, about 0.023 M, about 0.024 M, about 0.025 M, about 0.026 M, about 0.027 M, about 0.028 M, about 0.029 M, about 0.03 M, about 0.031 M, about 0.032 M, about 0.033 M, about 0.034 M, about 0.035 M, about 0.036 M, about 0.037 M, about 0.038 M, about 0.039 M, about 0.04 M, about 0.041 M, about 0.042 M, about 0.043 M, about 0.044 M, about 0.045 M, about 0.046 M, about 0.047 M, about 0.048 M, about 0.049 M, or about 0.05 M NaOH. In an embodiment, the third solution comprises about 0.01 M NaOH.

In an embodiment, the first solution comprises about 1% acetic acid and about 1% phosphoric acid; the second solution comprises about 50 mM Tris, and the third solution comprises 0.005-0.05 M NaOH. In an embodiment, the first solution comprises about 1% acetic acid and about 1% phosphoric acid; the second solution comprises about 50 mM Tris, and the third solution comprises 0.009-0.015 M NaOH. In an embodiment, the first solution comprises about 1% acetic acid and about 1% phosphoric acid; the second solution comprises about 50 mM Tris, and the third solution comprises about 0.005 M, about 0.006 M, about 0.007 M, about 0.008 M, about 0.009 M, about 0.01 M, about 0.011 M, about 0.012 M, about 0.013 M, about 0.014 M, about 0.015 M, about 0.016 M, about 0.017 M, about 0.018 M, about 0.019 M, about 0.02 M, about 0.021 M, about 0.022 M, about 0.023 M, about 0.024 M, about 0.025 M, about 0.026 M, about 0.027 M, about 0.028 M, about 0.029 M, about 0.03 M, about 0.031 M, about 0.032 M, about 0.033 M, about 0.034 M, about 0.035 M, about 0.036 M, about 0.037 M, about 0.038 M, about 0.039 M, about 0.04 M, about 0.041 M, about 0.042 M, about 0.043 M, about 0.044 M, about 0.045 M, about 0.046 M, about 0.047 M, about 0.048 M, about 0.049 M, or about 0.05 M NaOH.

In an embodiment, the first solution comprises about 1% acetic acid and about 1% phosphoric acid; the second solution comprises about 50 mM Tris at a pH of about 8; and the third solution comprises about 0.01 M NaOH.

In an embodiment, the method further comprises washing the protein A chromatography matrix with the second solution after the protein A chromatography matrix is washed with the third solution.

In an embodiment, the method further comprises washing the protein A chromatography matrix with a fourth solution comprising about 0.05-0.5 M NaOH. In an embodiment, the method further comprises washing the protein A chromatography matrix with a fourth solution comprising about 0.05 M, about 0.06 M, about 0.07 M, about 0.08 M, about 0.09 M, about 0.1 M, about 0.11 M, about 0.12 M, about 0.13 M, about 0.14 M, about 0.15 M, about 0.16 M, about 0.17 M, about 0.18 M, about 0.19 M, about 0.2 M, about 0.25 M, about 0.3 M, about 0.35 M, about 0.4 M, about 0.45 M, or about 0.5 M NaOH. In an embodiment, the method further comprises washing the protein A chromatography matrix with a fourth solution comprising about 0.1 M NaOH.

In an aspect, provided herein is a method of cleaning a protein A chromatography matrix that has previously been used to purify an Fc-containing protein, the method comprising: sequentially washing the protein A chromatography matrix with: a) a first solution comprising about 1% acetic acid and about 1% phosphoric acid; b) a second solution comprising about 50 mM Tris at a pH of about 8; c) a third solution comprising about 0.01 M NaOH, d) the second solution; and e) a fourth solution comprising about 0.1 M NaOH.

In an aspect, provided herein is a method of cleaning a protein A chromatography matrix that has previously been used to purify dulaglutide, the method comprising: sequentially washing the protein A chromatography matrix with: a) a first solution comprising about 1% acetic acid and about 1% phosphoric acid; b) a second solution comprising about 50 mM Tris at a pH of about 8; c) a third solution comprising about 0.01 M NaOH, d) the second solution; and e) a fourth solution comprising about 0.1 M NaOH.

In an embodiment, the protein A chromatography matrix is washed with 1-10 column volumes of the first solution. In an embodiment, the protein A chromatography matrix is washed with about 1, about 2, about 3, about 4, about 5, about 6, about 7, about 8, about 9, or about 10 column volumes of the first solution. In an embodiment, the protein A chromatography matrix is washed with 2-3 column volumes of the first solution. In an embodiment, the protein A chromatography matrix is washed with about 2, about 2.1, about 2.2, about 2.3, about 2.4, about 2.5, about 2.6, about 2.7, about 2.8, about 2.9, or about 3 column volumes of the first solution.

In an embodiment, the protein A chromatography matrix is washed with 1-10 column volumes of the second solution. In an embodiment, the protein A chromatography matrix is washed with about 1, about 2, about 3, about 4, about 5, about 6, about 7, about 8, about 9, or about 10 column volumes of the second solution. In an embodiment, the protein A chromatography matrix is washed with 1-2 column volumes of the second solution. In an embodiment, the protein A chromatography matrix is washed with about 1, about 1.1, about 1.2, about 1.3, about 1.4, about 1.5, about 1.6, about 1.7, about 1.8, about 1.9, or about 2 column volumes of the second solution.

In an embodiment, the protein A chromatography matrix is washed with 1-10 column volumes of the third solution. In an embodiment, the protein A chromatography matrix is washed with about 1, about 2, about 3, about 4, about 5, about 6, about 7, about 8, about 9, or about 10 column volumes of the third solution. In an embodiment, the protein A chromatography matrix is washed with 2-3 column volumes of the third solution. In an embodiment, the protein A chromatography matrix is washed with about 2, about 2.1, about 2.2, about 2.3, about 2.4, about 2.5, about 2.6, about 2.7, about 2.8, about 2.9, or about 3 column volumes of the third solution.

In an embodiment, the method does not include a static hold of the first solution. In an embodiment, the method does not include a static hold of the second solution. In an embodiment, the method does not include a static hold of the third solution. In an embodiment, the method does not include a static hold of the first, second, and third solution.

In an embodiment, the protein A chromatography matrix is washed with: 2-3 column volumes of the first solution; 1-2 column volumes of the second solution; and 2-3 column volumes of the third solution.

In an embodiment, the protein A chromatography matrix is washed with: about 3 column volumes of the first solution; about 1.1 column volumes of the second solution; and about 2.2 column volumes of the third solution.

In an embodiment, the method further comprises washing the protein A chromatography matrix with about 2-10 column volumes of the second solution after the protein A chromatography matrix is washed with the third solution. In an embodiment, the method further comprises washing the protein A chromatography matrix with about 1, about 2, about 3, about 4, about 5, about 6, about 7, about 8, about 9, or about 10 column volumes of the second solution after the protein A chromatography matrix is washed with the third solution. In an embodiment, the method further comprises washing the protein A chromatography matrix with about 2 column volumes of the second solution after the protein A chromatography matrix is washed with the third solution.

In an embodiment, the protein A chromatography matrix is washed with 1-10 column volumes of the fourth solution. In an embodiment, the protein A chromatography matrix is washed with about 1, about 2, about 3, about 4, about 5, about 6, about 7, about 8, about 9, or about 10 column volumes of the fourth solution. In an embodiment, the protein A chromatography matrix is washed with 2-3 column volumes of the fourth solution. In an embodiment, the protein A chromatography matrix is washed with about 2, about 2.1, about 2.2, about 2.3, about 2.4, about 2.5, about 2.6, about 2.7, about 2.8, about 2.9, or about 3 column volumes of the fourth solution.

In an embodiment, the method further comprises storing the protein A chromatography matrix in a solution comprising about 100 mM acetic acid, sodium acetate at a pH of about 4.

In an embodiment, the protein A chromatography matrix is washed with the first solution at a flow rate of about 100-400 cm/hr. In an embodiment, the protein A chromatography matrix is washed with the first solution at a flow rate of about 100 cm/hr, 110 cm/hr, 120 cm/hr, 130 cm/hr, 140 cm/hr, about 150 cm/hr, 160 cm/hr, 170 cm/hr, 180 cm/hr, 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, about 300 cm/hr, about 310 cm/hr, about 320 cm/hr, about 330 cm/hr, about 340 cm/hr, about 350 cm/hr, about 360 cm/hr, about 370 cm/hr, about 380 cm/hr, about 390 cm/hr, or about 400 cm/hr. In an embodiment, the protein A chromatography matrix is washed with the first solution at a flow rate of about 270 cm/hr.

In an embodiment, the protein A chromatography matrix is washed with the second solution at a flow rate of about 100-400 cm/hr. In an embodiment, the protein A chromatography matrix is washed with the second solution at a flow rate of about 100 cm/hr, 110 cm/hr, 120 cm/hr, 130 cm/hr, 140 cm/hr, about 150 cm/hr, 160 cm/hr, 170 cm/hr, 180 cm/hr, 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, about 300 cm/hr, about 310 cm/hr, about 320 cm/hr, about 330 cm/hr, about 340 cm/hr, about 350 cm/hr, about 360 cm/hr, about 370 cm/hr, about 380 cm/hr, about 390 cm/hr, or about 400 cm/hr. In an embodiment, the protein A chromatography matrix is washed with the second solution at a flow rate of about 270 cm/hr.

In an embodiment, the protein A chromatography matrix is washed with the third solution at a flow rate of about 100-400 cm/hr. In an embodiment, the protein A chromatography matrix is washed with the third solution at a flow rate of about 100 cm/hr, 110 cm/hr, 120 cm/hr, 130 cm/hr, 140 cm/hr, about 150 cm/hr, 160 cm/hr, 170 cm/hr, 180 cm/hr, 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, about 300 cm/hr, about 310 cm/hr, about 320 cm/hr, about 330 cm/hr, about 340 cm/hr, about 350 cm/hr, about 360 cm/hr, about 370 cm/hr, about 380 cm/hr, about 390 cm/hr, or about 400 cm/hr. In an embodiment, the protein A chromatography matrix is washed with the third solution at a flow rate of about 270 cm/hr.

In an embodiment, the column is washed with the first solution at a flow rate of about 270 cm/hr, the second solution at a flow rate of about 270 cm/hr, and the third solution at a flow rate of about 270 cm/hr.

In an embodiment, the protein A chromatography matrix is washed with about 2 column volumes of the fourth solution. In an embodiment, the protein A chromatography matrix is washed with the fourth solution at a flow rate of 100-140 cm/hr. In an embodiment, the protein A chromatography matrix is washed with the fourth solution at a flow rate of about 130 cm/hr. In an embodiment, the protein A chromatography matrix is washed with the fourth solution at a flow rate of 100-110 cm/hr. In an embodiment, the protein A chromatography matrix is washed with the fourth solution at a flow rate of about 100 cm/hr, about 101 cm/hr, about 102 cm/hr, about 103 cm/hr, about 104 cm/hr, about 105 cm/hr, about 106 cm/hr, about 107 cm/hr, about 108 cm/hr, about 109 cm/hr, or about 110 cm/hr. In an embodiment, the protein A chromatography matrix is washed with the fourth solution for 10-30 minutes. In an embodiment, the protein A chromatography matrix is washed with the fourth solution for about 10, about 15, about 20, about 25 minutes, or about 30 minutes.

In an embodiment, the protein A chromatography matrix is washed with the first solution, second solution, and/or third solution in an upflow or downflow direction. In an embodiment, the protein A chromatography matrix is washed with the first solution in an upflow direction. In an embodiment, the protein A chromatography matrix is washed with the second solution in an upflow direction. In an embodiment, the protein A chromatography matrix is washed with the third solution in an upflow direction. In an embodiment, the protein A chromatography matrix is washed with the fourth solution in an upflow direction.

In an aspect, any one of the methods disclosed herein reduces the carryover from the previous use of the protein A chromatography matrix. In an embodiment, the protein A chromatography matrix has less than about 1% carryover from the previous use after the protein A chromatography matrix is cleaned. In an embodiment, the protein A chromatography matrix has less than about 0.1, less than about 0.2, less than about 0.3, less than about 0.4, less than about 0.5, less than about 0.6, less than about 0.7, less than about 0.8, less than about 0.9, or less than about 1.0% carryover from the previous use after the protein A chromatography matrix is cleaned. In an embodiment, the protein A chromatography matrix has less than about 0.1% carryover from the previous use after the protein A chromatography matrix is cleaned.

In an embodiment, any one of the methods disclosed herein removes one or more residual contaminant from the protein A chromatography matrix. In an embodiment, the contaminant is TritonX-100, DNA, HCP, leached protein A, or insulin.

In an embodiment, the protein A chromatography matrix is used for 200-500 cycles of Fc-containing protein purification. In an embodiment, the protein A chromatography matrix is used for 300-400 cycles of Fc-containing protein purification. In an embodiment, the protein A chromatography matrix is used for about 300, about 301, about 302, about 303, about 304, about 305, about 306, about 307, about 308, about 309, about 310, about 311, about 312, about 313, about 314, about 315, about 316, about 317, about 318, about 319, about 320, about 321, about 322, about 323, about 324, about 325, about 326, about 327, about 328, about 329, about 330, about 331, about 332, about 333, about 334, about 335, about 336, about 337, about 338, about 339, about 340, about 341, about 342, about 343, about 344, about 345, about 346, about 347, about 348, about 349, about 350, about 351, about 352, about 353, about 354, about 355, about 356, about 357, about 358, about 359, about 360, about 361, about 362, about 363, about 364, about 365, about 366, about 367, about 368, about 369, about 370, about 371, about 372, about 373, about 374, about 375, about 376, about 377, about 378, about 379, about 380, about 381, about 382, about 383, about 384, about 385, about 386, about 387, about 388, about 389, about 390, about 391, about 392, about 393, about 394, about 395, about 396, about 397, about 398, about 399, or about 400 cycles of Fc-containing protein purification. In an embodiment, the protein A chromatography matrix is used for about 304 cycles of Fc-containing protein purification.

In an embodiment, the protein A chromatography matrix is used for 200-500 cycles of dulaglutide purification. In an embodiment, the protein A chromatography matrix is used for 300-400 cycles of dulaglutide purification. In an embodiment, the protein A chromatography matrix is used for about 300, about 301, about 302, about 303, about 304, about 305, about 306, about 307, about 308, about 309, about 310, about 311, about 312, about 313, about 314, about 315, about 316, about 317, about 318, about 319, about 320, about 321, about 322, about 323, about 324, about 325, about 326, about 327, about 328, about 329, about 330, about 331, about 332, about 333, about 334, about 335, about 336, about 337, about 338, about 339, about 340, about 341, about 342, about 343, about 344, about 345, about 346, about 347, about 348, about 349, about 350, about 351, about 352, about 353, about 354, about 355, about 356, about 357, about 358, about 359, about 360, about 361, about 362, about 363, about 364, about 365, about 366, about 367, about 368, about 369, about 370, about 371, about 372, about 373, about 374, about 375, about 376, about 377, about 378, about 379, about 380, about 381, about 382, about 383, about 384, about 385, about 386, about 387, about 388, about 389, about 390, about 391, about 392, about 393, about 394, about 395, about 396, about 397, about 398, about 399, or about 400 cycles of dulaglutide purification. In an embodiment, the protein A chromatography matrix is used for about 304 cycles of dulaglutide purification.

### Protein A Chromatography

The methods provided herein generally comprise washing a chromatography column comprising a protein A chromatography matrix.

In an embodiment, the chromatography column has a diameter of 10-150 cm. In an embodiment, the chromatography column has a diameter of about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 95 cm, about 100 cm, about 110 cm, about 120 cm, about 130 cm, about 140 cm, or about 150 cm.

In an embodiment, the chromatography column has a bed height of 10-40 cm. In an embodiment, the chromatography column has a bed height of about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 30.5 cm, about 31 cm, about 31.5 cm, about 32 cm, about 32.5 cm, about 33 cm, about 33.5 cm, about 34 cm, about 34.5 cm, about 35 cm, about 35.5 cm, about 36 cm, about 36.5 cm, about 37 cm, about 37.5 cm, about 38 cm, about 38.5 cm, about 39 cm, about 39.5 cm, or about 40 cm.

In an embodiment, the chromatography column is loaded at a temperature of about 10-40° C. In an embodiment, the chromatography column is loaded at a temperature of about 15-35° C. In an embodiment, the chromatography column is loaded at a temperature of about 15-30° C.

In an embodiment, the protein A chromatography matrix has an average particle size of 80-90 μm. In an embodiment, the protein A chromatography matrix has an average particle size of about 80 μm, about 81 μm, about 82 μm, about 83 μm, about 84 μm, about 85 μm, about 86 μm, about 87 μm, about 88 μm, about 89 μm, or about 90 μm. In an embodiment, the protein A chromatography matrix has an average particle size of about 85 μm.

In an embodiment, the protein A chromatography matrix comprises a protein A ligand that has increased stability under alkali conditions relative to wild type Staphylococcus aureus protein A. In an embodiment, the protein A chromatography matrix comprises an engineered variant of protein A that is more stable in alkali than wild-type protein A. In an embodiment, the protein A chromatography matrix comprises an engineered variant of protein A that is modified to substitute particular amino acids that are sensitive to alkali with amino acids that are more stable in alkali.

In an embodiment, the protein A chromatography matrix comprises a protein A ligand that has increased stability under alkali conditions relative to wild type Staphylococcus aureus protein A and has an average particle size of about 85 μm.

Various protein A chromatography matrices are available that are suitable for use in the methods disclosed herein. The protein A chromatography matrices can have various backbone compositions including, for example, glass or silica-based matrices, agarose-based matrices, and organic polymer-based matrices. In an embodiment, the protein A chromatography matrix comprises an engineered variant of protein A. In an embodiment, the protein A amino acid sequence comprises a C-terminal cysteine for cross-linking to a matrix. In an embodiment, the protein A chromatography matrix is an agarose matrix. In an embodiment, the protein A chromatography matrix comprises protein A tetramers cross-linked to the agarose matrix via the C-terminal cysteine on protein A. In an embodiment, the protein A chromatography matrix comprises protein A tetramers cross-linked to the agarose matrix via an epoxide linkage.

In an embodiment, the protein A chromatography matrix is a MabSelect™ protein A chromatography matrix from Cytiva (Marlborough, MA). In an embodiment, the MabSelect™ protein A chromatography matrix is MabSelect SuRe™, MabSelect SuRe™ LX, MabSelect SuRe™ pcc, or MabSelect PrismA™.

In an embodiment, provided herein is a method of cleaning MabSelect SuRe™ LX that has been used to purify an Fc-containing protein, such that the MabSelect SuRe™ LX can be used for about 304 cycles of purifying the Fc-containing protein. In an embodiment, provided herein is a method of cleaning MabSelect SuRe™ LX that has been used to purify dulaglutide, such that the MabSelect SuRe™ LX can be used for about 304 cycles of purifying the dulaglutide.

In an aspect, provided herein is a method of purifying an Fc-containing protein using a protein A chromatography matrix that has been cleaned according to any one of the methods disclosed herein.

In an aspect, provided herein is a method of purifying dulaglutide using a protein A chromatography matrix that has been cleaned according to any one of the methods disclosed herein.

### III. Fc-Containing Proteins

The methods provided by the present disclosure are for the purification of an Fc-containing protein from a mixture of the Fc-containing protein and one or more contaminant.

In an embodiment, the Fc-containing protein was produced in mammalian host cells. In an embodiment, the Fc-containing protein was produced in Chinese Hamster Ovary (CHO) cells, baby hamster kidney (BHK) cells, murine hybridoma cells, HEK cells, or murine myeloma cells.

In an embodiment, the Fc-containing protein comprises one or more of the amino acid sequences set forth in Table 1 below.

In an embodiment, the Fc-containing protein comprises a glucagon-like peptide 1 (GLP-1) analog comprising one or more modifications compared to a wild type GLP-1 amino acid sequence (SEQ ID NO: 1).

In an embodiment, the Fc-containing protein comprises a GLP-1 analog comprising the amino acid sequence of SEQ ID NO: 2.

In an embodiment, the Fc-containing protein comprises a peptide linker. In an embodiment, the C-terminal amino acid of the GLP-1 analog portion of the Fc-containing protein is fused to the N-terminus of an IgG₄ Fc analog portion via a glycine-rich linker. In an embodiment, the peptide linker comprises 1 to 10 G₄S units (SEQ ID NO: 6).

In an embodiment, the Fc-containing protein comprises: a GLP-1 analog comprising the amino acid sequence of SEQ ID NO: 2; a peptide linker comprising 1 to 10 G₄S units (SEQ ID NO: 6); and an Fc portion of an immunoglobulin. In an embodiment, the N-terminal residue of the peptide linker is directly fused to the C-terminal residue of the GLP-1 analog, and the C-terminal residue of the peptide linker is directly fused to the N-terminal residue of the Fc portion.

In an embodiment, the Fc-containing protein comprises the amino acid sequence of SEQ ID NO: 5. In an embodiment, the wherein the Fc-containing protein is a homodimer comprising two identical amino acid chains each comprising the amino acid sequence of SEQ ID NO: 5. Fc-containing protein comprises a homodimer of the amino acid sequence of SEQ ID NO: 5.

In an embodiment, the Fc-containing protein is dulaglutide.

In an aspect, provided herein are methods for the purification of dulaglutide from a mixture of dulaglutide and one or more HCP. In an embodiment, dulaglutide is produced in CHO cells.

Dulaglutide is a human GLP-1 receptor agonist which comprises a dimer of a GLP-1 analog fused at its C-terminus via a (G₄S)₃ peptide linker to the N-terminus of an analog of an Fc portion of an immunoglobulin, and is identified by CAS registry number 923950-08-7, which provides the following chemical name: 7-37-Glucagon-like peptide I [8-glycine, 22-glutamic acid, 36-glycine] (synthetic human) fusion protein with peptide (synthetic 16-amino acid linker) fusion protein with immunoglobulin G4 (synthetic human Fc fragment), dimer. Each monomer of dulaglutide has the amino acid sequence set forth in SEQ ID NO: 5.

The two monomers are attached by disulfide bonds between the cysteine residues at positions 55 and 58 of SEQ ID NO: 5 to form the dimer. Dulaglutide's structure, function, production, and use in treating T2DM is described in more detail in U.S. Pat. No. 7,452,966 and U.S. Patent Application Publication No. US20100196405. Dulaglutide agonizes the GLP-1 receptor resulting in stimulation of insulin synthesis and secretion and has been shown to provide improved glycemic control in T2DM patients.

When used herein, the term “dulaglutide” refers to any GLP-1 receptor agonist protein dimer of two monomers having the amino acid sequence of SEQ ID NO: 5, including any protein that is the subject of a regulatory submission seeking approval of a GLP-1 receptor agonist product which relies in whole or part upon data submitted to a regulatory agency by Eli Lilly and Company relating to dulaglutide, regardless of whether the party seeking approval of said protein actually identifies the protein as dulaglutide or uses some other term.

TABLE 1
Sequences comprised in certain
Fc-containing proteins

| | SEQ ID | |
|---|---|---|
| Description | NO: | Amino acid sequence |
| WT GLP 1 | 1 | HAEGTFTSDVSSYLEGQAAKEFIAWLVKGR |
| | | G |
| GLP 1 | 2 | HGEGTFTSDVSSYLEEQAAKEFIAWLVKGG |
| | | G |
| variant | | |
| G₄S | 6 | GGGGS |
| Dulaglutide | 5 | HGEGTFTSDVSSYLEEQAAKEFIAWLVKGG |
| monomer | | GGGGGSGGGGSGGGGSAESKYGPPCPPCPA |
| | | PEAAGGPSVFLFPPKPKDTLMISRTPEVTC |
| | | VVVDVSQEDPEVQFNWYVDGVEVHNAKTKP |
| | | REEQFNSTYRVVSVLTVLHQDWLNGKEYKC |
| | | KVSNKGLPSSIEKTISKAKGQPREPQVYTL |
| | | PPSQEEMTKNQVSLTCLVKGFYPSDIAVEW |
| | | ESNGQPENNYKTTPPVLDSDGSFFLYSRLT |
| | | VDKSRWQEGNVFSCSVMHEALHNHYTQKSL |
| | | SLSLG |

In an embodiment, the Fc-containing protein is etanercept, alefacept, abatacept, rilonacept, romiplostim, belatacept, aflibercept, conbercept, efmoroctocog alpha, eftrenonacog alpha, asfotase alpha, or luspatercept.

In an embodiment, the Fc-containing protein is an antibody. In an embodiment, the Fc-containing protein is not an antibody.

### EXAMPLES

The following examples are offered by way of illustration, and not by way of limitation.

### Example 1: Protein a Chromatography Matrix Lifetime Study

In the protein A purification step of the dulaglutide manufacturing process, the protein A chromatography matrix is re-used up to 112 purification cycles with a cleaning protocol that includes a wash with an acid solution (1% acetic acid/1% phosphoric acid) to regenerate the matrix and a wash with a solution containing 50 mM NaOH and 1 M NaCl to sanitize the matrix. In this example, a new method of cleaning the protein A chromatography matrix was analyzed for the purpose of extending the usable lifetime of the protein A chromatography matrix and to establish a maximum number of dulaglutide purification cycles the protein A chromatography matrix can be used for.

As an initial test to determine the importance of the first acid wash in the cleaning protocol after dulaglutide purification, a protein A chromatography matrix (MabSelect SuRE™ LX) was cleaned in place with only a caustic solution containing 0.05 M NaOH for 4 successive runs, in the absence of a first step using an acid solution. The results from this test demonstrated that the NaOH wash alone resulted in increasing column pressure after each successive run. These initial results indicated that an acid wash is needed in the cleaning protocol for the protein A chromatography matrix after dulaglutide purification to control column pressure.

Further, a previous protein A chromatography lifetime study for dulaglutide purification resulted in a run-on-run increase in pre-peak size. In order to better control this increase, a caustic wash was introduced in the regeneration step of the cleaning protocol subsequent to the acid wash step, using 0.01 M NaOH for 2 column volumes.

The study described below provides results for an improved cleaning protocol of MabSelect SuRe™ LX after dulaglutide purification that includes an additional wash with a caustic solution (0.01 M NaOH) between the acid wash (1% acetic acid/1% phosphoric acid) and the sanitization wash.

### Methods

The protein A chromatography matrix cleaning protocol for this study included a wash with an acid solution containing 1% acetic acid, 1% phosphoric acid, without a static hold, followed by a wash with a Tris equilibration buffer (50 mM Tris, pH 8), then a wash with a caustic solution (0.01 M NaOH). The protein A chromatography matrix was then sanitized by washing the matrix again with the Tris equilibration buffer followed by a wash with a solution containing 0.1 M NaOH. The general parameters for the protein A chromatography matrix cleaning study are described in Table 4 below. MabSelect SuRe™ LX, which has a protein A ligand that is engineered to have increased stability in alkali conditions, was used for the study.

TABLE 4
Operating parameters for protein A dulaglutide
purification lifetime study.

| | | Target | Target Flow | |
|---|---|---|---|---|
| | | Volume | Velocity | |
| Step | Buffer/Parameter | (CV) | (cm/hr) | Direction |
| Pre-use | 50 mM Tris pH 8.0 | 1.1 | 270 | UP |
| Sani | 0.1N NaOH | 2.0² | 103^{1, 2} | UP |
| | 50 mM Tris pH 8.0 | 2.0 | 103^{1, 2} | UP |
| Column | 50 mM Tris pH 8.0 | 2.2 | 270 | DOWN |
| Equilibration | | | | |
| Charge | | | 150 | DOWN |
| Washes | 50 mM Tris pH 8.0 | 5.0 | 300 | DOWN |
| | 50 mM Tris pH 8.0 | 5.0 | 300 | DOWN |
| | 1M NaCl | | | |
| | 50 mM Tris pH 8.0 | 5.0 | 300 | DOWN |
| | 10 mM Sodium | 5.0 | 270 ³ | DOWN |
| | Citrate, | | | |
| | pH 3.0 | | | |
| | FS Cut UV (4 AU/cm) | | | |
| | BS Cut (CV) | 1.5 ⁴ | | |
| | Elution Buffer | 0.6 | | |
| | Pre-Load (CV) | | | |
| Column | 1% Acetic Acid, 1% | 2.2 ⁵ | 270 | UP |
| Regeneration | Phosphoric Acid | | | |
| | 50 mM Tris pH 8.0 | 1.1 | 270 | UP |
| | 0.01N NaOH | 2.2 | 270 | UP |
| | 50 mM Tris pH 8.0 | 2.0 | 270 | UP |
| Post-use | 50 mM Tris pH 8.0 | 1.1 | 270 | UP |
| Sani | 0.1N NaOH | 2.0² | 103^{1, 2} | UP |
| | 50 mM Tris pH 8.0 | 2.0 | 103^{1, 2} | UP |
| Storage | 100 mM Acetic Acid, | 2.2 | 270 | UP |
| | Sodium Acetate, | | | |
| | pH 4.0 | | | |

¹103 cm/hr post re-pack as column size reduced, runs 1-88 flowrate was set at 107 cm/hr.
²Target volumetric endpoint and flowrate differ from current manufacturing protocols, however, the target contact time of 25 mins is equivalent.
³ Elution flowrate used in current manufacturing protocols is 210 cm/hr, flowrate of 270 cm/hr used in this study is within the parameter range of 200-330 cm/hr.
⁴ BS Cut used in current manufacturing protocols is set at 1.76 CV, 1.5 CV target (within range 1.43-1.76 CV) more appropriate for lab-scale model due to elution profile differences at-scale.
⁵ Acid regeneration step in current manufacturing protocols is set to 3 CV of 1% acetic acid, 1% phosphoric acid.

### Column Packing and Evaluation

The protein A chromatography matrix, MabSelect SuRe LX (MSS LX) was packed into a Millipore Vantage Pro 1.1 cm column using an AKTA Avant 25 liquid handling module (MST367). A secondary column was used to accommodate the matrix slurry. The ~50% slurry of MSS LX in 20% EtOH 400 mM NaCl was introduced to the open column top; the head was then attached, with consolidation of the resin slurry at 2× the maximum process flowrate. Upon consolidation, the secondary column was removed. The column top adaptor was positioned on top of the resin bed with flow reapplied to the bed. When no further drop in bed height was seen the column was considered packed. Finally, the column was conditioned post packing in the packing buffer which was passed through the column at operational flowrate for 5 column volumes (CV). The column was packed to a bed height of 22.3 cm, within the range of 20-32 cm, as per the dulaglutide purification process.

The packed column was evaluated for suitability for use by measuring height equivalent to theoretical plate (HETP) and Asymmetry. All column pack evaluations (HETP and Asymmetry method) were completed with the same conditions throughout the study and matched that of large-scale practices where possible. The column was equilibrated with 100 mM NaCl to achieve a conductivity baseline. The pulse test solution was IM NaCl with an injection volume of 2% of the column volume. The column was equilibrated at 100 cm/hr and the injection and elution flow rates were at 100 cm/hr. A column pack with virgin resin is suitable for use if the theoretical plate count is ≥1100 plates/m and the asymmetry value between 0.7 and 1.8.

### Column Functional Assessment

The packed column was sanitized and stored post column pack evaluation. Following this storage, a column functional assessment was performed, consisting of a mock elution and dynamic binding capacity (DBC) assessment. See Tables 5 and 6 below for operating parameters for the mock elution and DBC assessment, respectively.

TABLE 5
Mock elution parameters for protein A resin lifetime study.

| | | Target | Flow | |
|---|---|---|---|---|
| | | Volume | Velocity | |
| Step | Buffer/Parameter | (CV) | (cm/hr) | Direction |
| Column | 50 mM Tris pH 8.0 | 2.2 | 270 | DOWN |
| Equilibration | | | | |
| Column | 50 mM Tris pH 8.0 | 5.0* | 300* | DOWN |
| Washes | 50 mM Tris pH 8.0, | 5.0 | 300 | DOWN |
| | 1M NaCl | | | |
| | 50 mM Tris pH 8.0 | 5.0 | 300 | DOWN |
| Column | 10 mM Sodium | 5.0 | 270 | DOWN |
| Elution | Citrate, | | | |
| | pH 3.0 | | | |
| | FS Cut (AU/cm) | | | |
| | 4 AU/cm | | | |
| Column | 1% Acetic Acid, | 2.2 | 270 | UP |
| Regeneration | 1% Phosphoric Acid | | | |
| | 50 mM Tris pH 8.0 | 1.1 | 270 | UP |
| | 0.01N NaOH | 2.2 | 270 | UP |
| | 50 mM Tris pH 8.0 | 2 | 270 | UP |

*Runs 65-80 Wash 1 flow rates were amended to reflect changes implemented for dulaglutide processing. These changes constituted moving from 300 cm/hr for 5.0 CVs to 150 cm/hr for the first 3.2 CVs followed by 300 cm/hr for 2.2 CVs.

TABLE 6
Dynamic binding capacity (DBC) parameters
for protein A resin lifetime study.

| | | Target | Flow | |
|---|---|---|---|---|
| | | Volume | Velocity | |
| Step | Buffer/Parameter | (CV) | (cm/hr) | Direction |
| Column | 50 mM Tris pH 8.0 | 2.2 | 270 | DOWN |
| Equilibration | | | | |
| Charge | | | 150 | DOWN |
| Column | 10 mM Sodium Citrate, | 5.0 | 270 | DOWN |
| Elution | pH 3.0 | | | |
| Column | 1% Acetic Acid, | 2.2 | 270 | UP |
| Regeneration | 1% Phosphoric Acid | | | |
| | 50 mM Tris pH 8.0 | 1.1 | 270 | UP |
| | 0.01N NaOH | 2.2 | 270 | UP |
| | 50 mM Tris pH 8.0 | 2.0 | 270 | UP |
| Post-use | 0.1N NaOH | 2.0 | 103* | UP |
| Sani | 50 mM Tris pH 8.0 | 1.1 | 103* | UP |
| Storage | 100 mM Acetic Acid, | 2.2 | 270 | UP |
| | Sodium Acetate, | | | |
| | pH 4.0 | | | |

The mock elution mimics conditions that the protein A chromatography matrix will be exposed to during dulaglutide purification, including column equilibration, washes, elution and cleaning and post-use sanitization. The blank peak carryover was then calculated as a percent of the total peak area of eluted dulaglutide in the prior cycle based on an A280 pathlength of 2 mm and using the equation below:

$$\frac{Mock Elution Peak Area ( ml * mAU )}{Prior Cycle Elution Peak Area ( ml * mAU )} \times 100 = \% Blank Carryover$$

The DBC assessment provides a read-out (QB10) of the dynamic binding capacity of the resin. QB10 was determined using the equation below:

$$Q {B}_{10} = ( {V}_{10 \%} - {V}_{s} - {V}_{void} ) \times C / {V}_{c}$$

In the above equation, V_{10%}=volume at 10% breakthrough; VS=volume at start of sample application; V_{void}=AKTA flowpath void volume (equivalent to column volume on AKTA systems); C=Concentration of sample solution to 3 digits accuracy; V_{C}=column volume. (V_{10%}−V_{S}) was determined through Unicorn functions.

### Protein A Affinity Chromatography

Protein A affinity chromatography was performed using a column packed with MSSLX matrix. The matrix was charged with a dulaglutide detergent viral inactivation (DVI) intermediate that had been stored at least at −65° C. Charge material aliquots were thawed and brought to room temperature prior to use and one 1 L aliquot was utilized for 4 product cycles each day. The final product cycle of the first, second and fourth days of each block were subjected to the low pH viral inactivation (LpHVI) incubation time (i.e., Runs 4, 8 and 16) and the rest were immediately neutralized. DVI batches used were rotated throughout the study. Dulaglutide DVI intermediate was loaded within the proven acceptable range (PAR) of 10-18 g/L to evaluate parametric data across the resin lifetime.

After thawing and bringing to room temperature in a water bath set at 23° C., material was 0.2 μm PES-filtered through a vacutainer. All runs were loaded at 14.4 g/L except for 2 runs from each block, run 8 which was charged at 18 g/L (upper PAR loading) and run 16 charged at 10 g/L (lower PAR loading). Runs 145-148 were charged at incorrect loadings as the charge batch was originally assigned a concentration of 1.62358 g/L. Following low yields in this study, this batch was re-tested and assigned a corrected concentration of 1.439226 g/L. The recalculated loading, which was applied based on the corrected concentration of 1.439226 g/L, was calculated to be 12.8 g/L and is not considered to have impacted the study as it remained within the PAR for loading (10-18 g/L).

The targeted resin lifetime for MSS LX resin was 304 product runs. A total of 304 product runs were completed using the MSS LX resin with analytical testing undertaken (rHCP, rProA and SEC) on the protein A mainstream for the first, fourth, eighth and sixteenth cycles of Blocks 1-19. Further testing (insulin, rDNA and Triton X-100) was performed on the eighth cycle (upper PAR load) from every second block for the duration of the lifetime.

### Column Pack Evaluation

Criteria for Height Equivalent to Theoretical Plate (HETP) and Asymmetry (i.e., column asymmetry) are presented in Table 7 below. These criteria are critical for determining the acceptable performance of the protein A chromatography resin.

TABLE 7
Column Pack Evaluation Acceptance Criteria

| Attribute | Acceptance Criteria |
|---|---|
| HETP (plates/m) | ≥1100 |
| Asymmetry | 0.7-1.8 |

### Column Performance Evaluation

Protein A/low pH viral inactivation (LpHVI) % yield must remain within the 70-105% IPC. During the study the following parameters were evaluated to assess consistent performance of the protein A chromatography matrix: yield, mainstream pH, column pack quality, mock elution carryover, dynamic binding, column outlet A280, pH, conductivity, and column delta pressure.

### Critical Quality Attributes

Product purity is routinely tested at the low pH viral inactivation step during dulaglutide manufacture, as such, the product validation acceptance criteria at low pH viral inactivation is also used in this study. In the case of rDNA, this is normally tested post the AEX unit operation, and as such the AEX acceptance criteria is used in this study as a representative limit. See Table 8 below for acceptance criteria applied throughout study. Results were evaluated with consideration for the applicable low pH viral inactivation acceptance criteria and historical small and/or large-scale experience. rHCP=residual host cell protein; rDNA=residual deoxyribonucleic acid; rProA=residual protein A.

TABLE 8
Process validation acceptance criteria for dulaglutide intermediates.

| | Unit | Process Validation |
|---|---|---|
| Test | Operation | Acceptance Criteria |
| rHCP (ppm) | LpHVI | ≤482 |
| rDNA (ppb) | AEX | *≤500 |
| rTriton X-100 (ppm) | LpHVI | ≤518000 |
| SEC Monomer % | LpHVI | ≥91.2 |
| Residual Insulin (μU/ml) | LpHVI | ≤20 |
| rProA (ppm) | LpHVI | ≤523 |

*Post AEX unit operation, no acceptance criteria defined for low pH viral inactivation (LpHVI)

### Results

### Column Performance

Target column loading for this study was 14.4 g/L. All runs met this target except for runs 145-148 where a DVI concentration error resulted in a reduced charge of 12.8 g/L. There is no impact to the study conclusions as the loads for all runs were within acceptable range for loading of 10-18 g/L, as discussed above. One run from each block targeted the upper limit of the proven acceptable range for column loading for the protein A unit operation of 18 g/L and one targeted the lower limit of 10 g/L.

Elution peak morphology was consistent across the column loading range. The elution pre-peak, observed before the elution peak is absent in runs loaded at the lower column load of 10 g/L while the pre-peak is larger in higher column loads of 18 g/L. This elution pre-peak was observed to grow across the lifetime of the study from 0.211 AU/cm to 0.468 AU/cm at the upper proven acceptable range loading, most likely due to a combination of fouling and matrix ligand hydrolysis. The frontside cut is triggered at a UV signal of 4 AU/cm, indicating that at the upper limit of the proven acceptable range for loading pre-peak was not in danger of triggering early elution as seen in previous studies, and is evidence towards the effectiveness of the protein A chromatography matrix cleaning step used in the present study.

No peak broadening with the exception of load-dependent differences was observed across the lifetime of the column while pH transitions and delta column pressures remained consistent. Broader peaks were observed for cycles run at the upper loading of 18 g/L and conversely narrower peaks at the lower loading of 10 g/L. These differences caused by loading also impacted the percentage yield of these cycles with higher column loads resulting in lower percentage yields. This was a result of a lower percentage of mainstream collected from broader peaks due to the backside cut occurring at a set volume (1.5 CV). Variability and tailing observed on the main elution peak, occurred randomly throughout the study and is likely a result of charge batch variation.

### pH and Conductivity

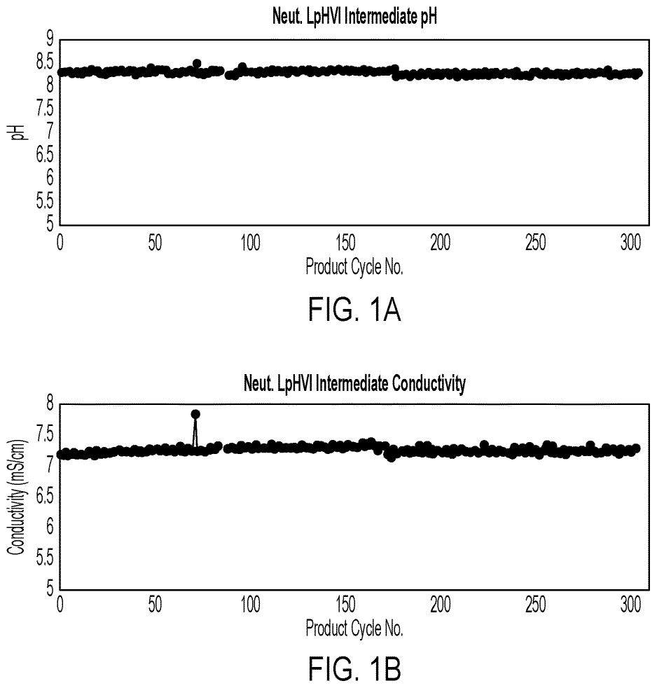

Off-line pH and conductivity values for charge and neutralized (neut.) low pH viral inactivation were monitored for all runs. The values for conductivity were read with the offline probe utilizing temperature compensation to 25° C. to normalize values. The low pH viral inactivated and neutralized mainstream pH response range observed in the lifetime study was 8.21 to 8.48 (average: pH 8.29±0.04) (FIG. 1A). Neutralized low pH viral inactivation conductivity was consistent across the lifetime study with a response range of 7.14-7.85 μS/cm (average: 7.27±0.06 μS/cm) (FIG. 1B). High pH and conductivity values were recorded at cycle 72 due to an error in neutralization buffer addition.

### Percent Yield (% Yield) and Dynamic Binding Capacity (DBC)

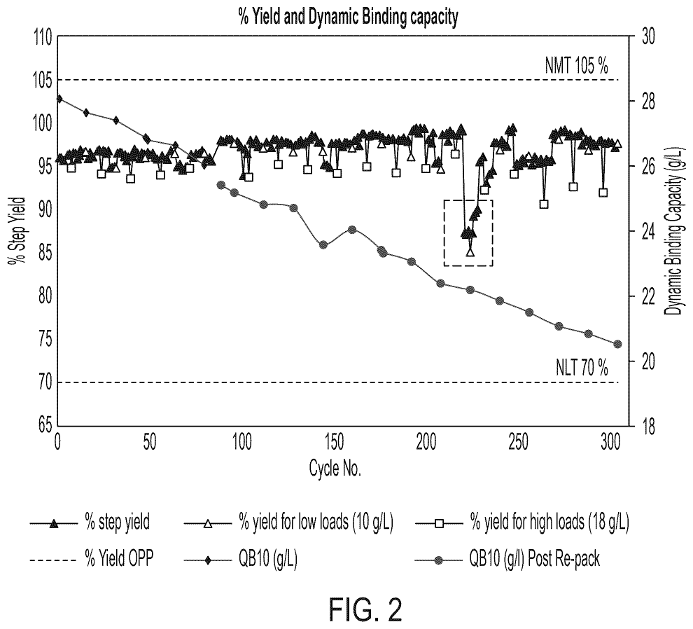

The cut strategy for dulaglutide protein A purification is based on a UV trigger and set collection volume. The frontside cut occurs at a UV signal of 4 AU/cm at 280 nm, and the backside cut in this study 1.5 CV after the frontside cut is made. FIG. 2 shows the % yield trend and reduction in dynamic binding capacity across the protein A resin lifetime. The % yield was calculated based on the concentration of the neut. low pH viral inactivation intermediate and the response range observed in the protein A cycle lifetime study was 84.4 to 99.6%. The yield data show that this parameter remained consistent across the resin lifetime with the exception of a hardware pump issue (Runs 221-236) (FIG. 2, red box, a pump malfunction resulting in reduced charge material loaded onto column but had no impact on the study conclusions). A small downward trend when accounting for batch variation was observed in the % yield towards the end of the lifetime on runs targeting the upper loading limit of 18 g/L. This is likely due to degradation of the MSS LX protein A ligand which is caustic liable and/or peak broadening at the upper proven acceptable range loading. However, the % yield across the lifetime remained well within the 70-105% critical in-process control (CIPC) range for the duration of the study.

The DBC observed in the protein A cycle lifetime study was 28.1 to 20.5 g/L. Based on these data, the protein A DBC decreased at a rate of 0.025 g/L per column cycle, most likely due to a combination of caustic sanitization exposure and binding site occlusion due to low level matrix fouling. Based on the equation of the slope of DBC values across the resin lifetime the DBC of the resin can be extrapolated to reach the upper proven acceptable range loading of 18 g/L after 408 product cycles. With a DBC value of 20.5 g/L at the end of this resin lifetime study and an anticipated routine manufacturing load of 14.4 g/L, this study demonstrates sufficient capacity of the resin to bind dulaglutide at the target load ratio over 304 product cycles.

### Column HETP and Asymmetry

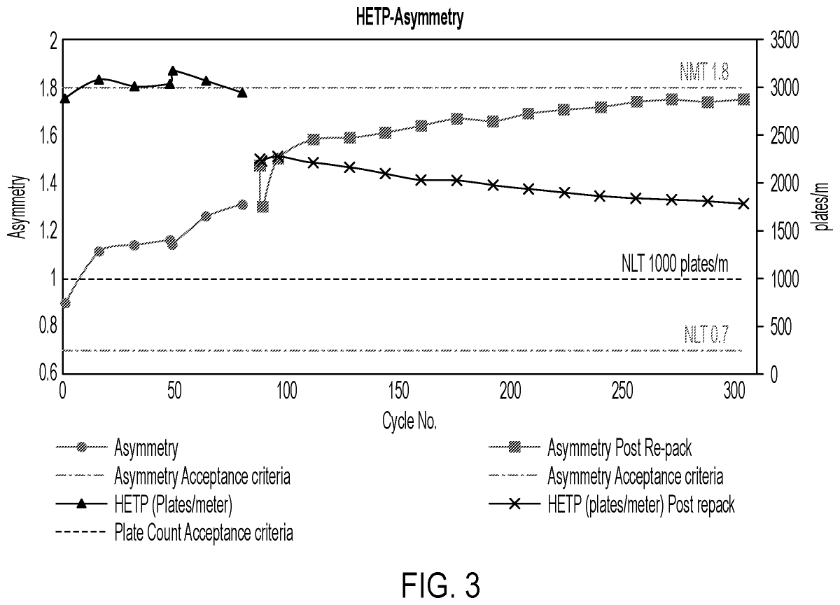

To detect changes in column pack quality, regular assessment of plates/meter and column asymmetry was performed across the column cycle lifetime study. The HETP observed in the protein A cycle lifetime study was 1782 to 3180 plates/meter. This range appears wider as the column repack performed post cycle 88 reduced the height of the column and the theoretical plate count but subsequently only a gentle decrease in plate number was observed which is anticipated due to repeat cycling. The plate count remained above the post-pack acceptance criteria of NLT 1100 plates/meter for the duration of the column lifetime study (FIG. 3).

The asymmetry observed was in the range of 0.90 to 1.75. An asymmetry value of 0.97 was observed on the initial column pack using virgin resin, which then increased to 1.30 following a column repack post cycle 88. The asymmetry gradually increased across the following 216 product cycles ending at 1.75 (FIG. 3) and this increase was not accompanied by any additional trends, with the exception of elution peak broadening at the upper limit of the proven acceptable range for loading which could potentially impact yields at this loading.

### Mock Elution Carryover

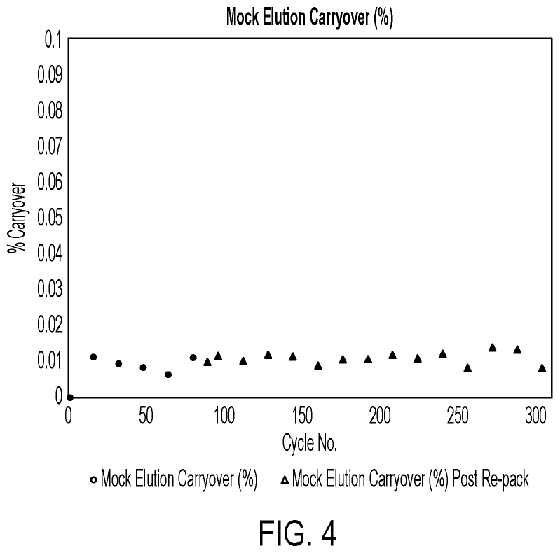

To detect potential changes in elution carryover, mock elutions were executed at regular intervals throughout the duration of this study. The mock elution percent carryover range observed in this protein A cycle lifetime study was 0 to 0.01% (FIG. 4) remaining below the <1% guideline limit for protein percent carryover.

### Product Purity by Size Exclusion Chromatography (SEC)

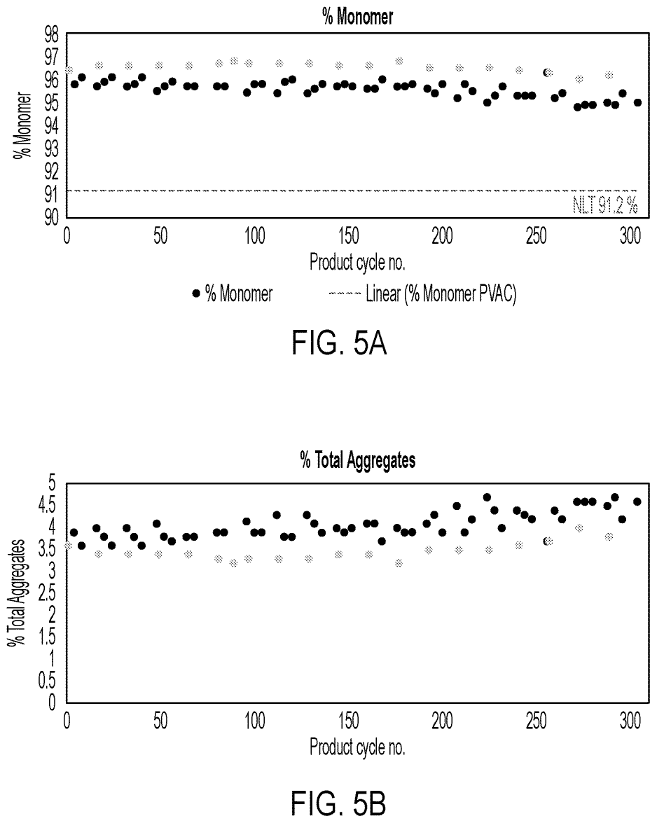

FIG. 5A-B shows that percent monomer and percent total aggregates all remained within acceptable levels throughout the duration of this study. Analytical testing by SEC during dulaglutide manufacture is carried out on the neutralized low pH viral inactivation (nLpHVI) intermediate with acceptance criteria of ≥91.2%. The data trend shows the percent monomer remained near constant across all column loadings for the duration of the first column pack (cycles 1-88). After the column re-pack the data trend remained steady until cycle 200 after which a slight (~0.5%) decrease in % monomer was observed across the remainder of the lifetime (FIG. 5A). As anticipated, the inclusion of incubation time for low pH viral inactivation is associated with minor aggregate growth which consequently results in lower % monomer, however this offset is maintained throughout the lifetime and is not impacted by column load or cycle number.

Residual Host Cell Proteins (rHCP)

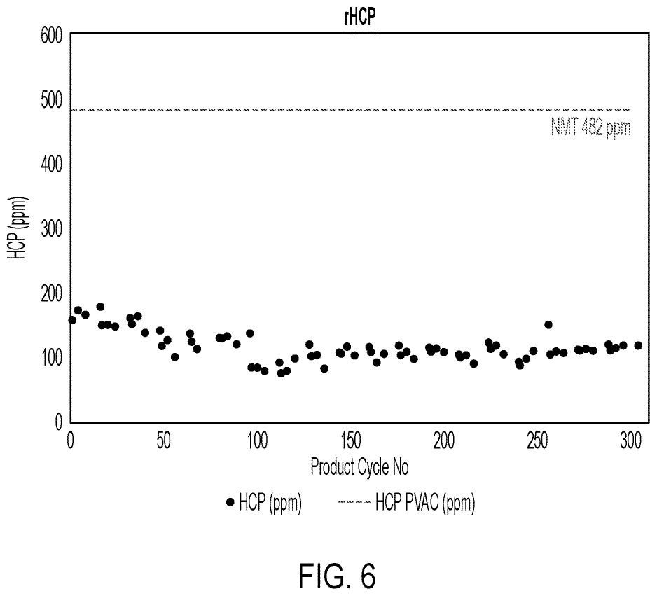

FIG. 6 shows that the neutralized low pH viral inactivation rHCP concentration (ppm) declined steadily for the first 100 cycles which is possibly due to the early cycling of virgin matrix. The column was repacked at cycle 88 with a slight reduction in bed height, after which rHCP levels remained consistent throughout the duration of this study meeting the ≤482 ppm process validation acceptance criteria set at low pH viral inactivation. This confirms the capability of the protein A chromatography matrix to clear process related impurities throughout the 304 cycles of the lifetime study.

Residual Protein A (rProA)

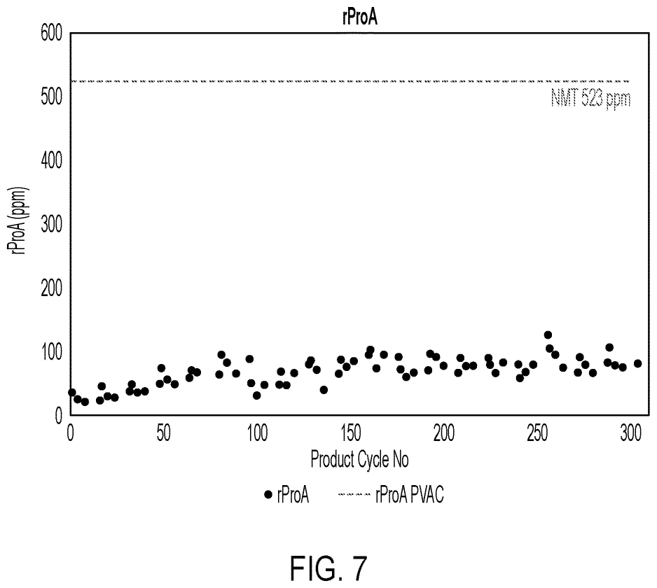

In order to assess the impact of increased caustic exposure on protein A leaching, the neutralized low pH viral inactivation was assessed for rProA. FIG. 7 shows that rProA levels (ppm) in the protein A mainstream displayed an upward trend at the beginning of the study for the duration of the first column pack. After the column was re-packed the rProA levels plateaued and remained consistent for the remainder of this study with a maximum value of 126 ppm. All samples taken across the PAR loading range of 10-18 g/L over the entire 304 cycles of this study met the ≤523 ppm process validation acceptance criteria at low pH viral inactivation. This confirms the capability of the resin to maintain acceptable levels of rProA across 304 product cycles.

Residual DNA (rDNA)

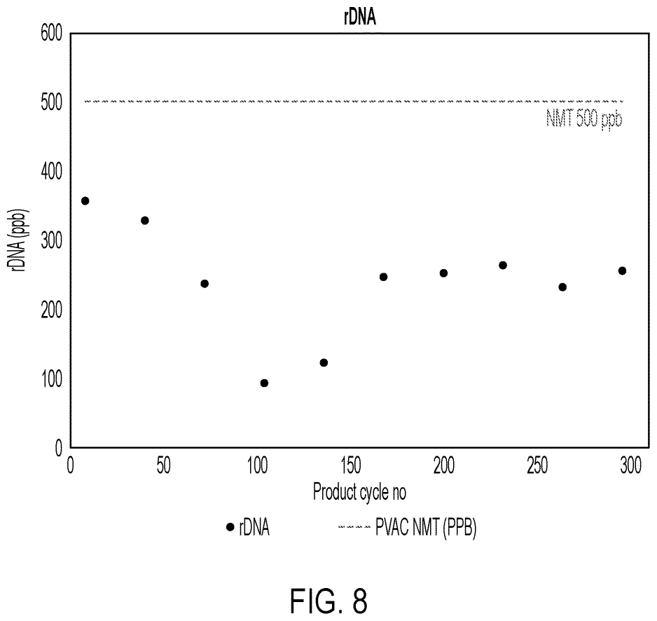

FIG. 8 shows that rDNA levels (ppb) in the protein A mainstream decreased steadily after the initial column pack. After the column was re-packed at cycle 88, rDNA levels initially increased before levelling off at ~250 ppb for the remainder of the lifetime. All samples were taken at the upper PAR load of 18 g/L, representative of a worst-case challenge, and over the entire 304 cycles of this study met the ≤500 ppb process validation acceptance criteria at AEX. This process validation acceptance criteria limit was used for representative purposes as there is no limit defined for rDNA at low pH viral inactivation. This confirms the capability of the resin to reduce rDNA to acceptable levels across 304 product cycles.

Residual Triton X-100 (rTX-100)

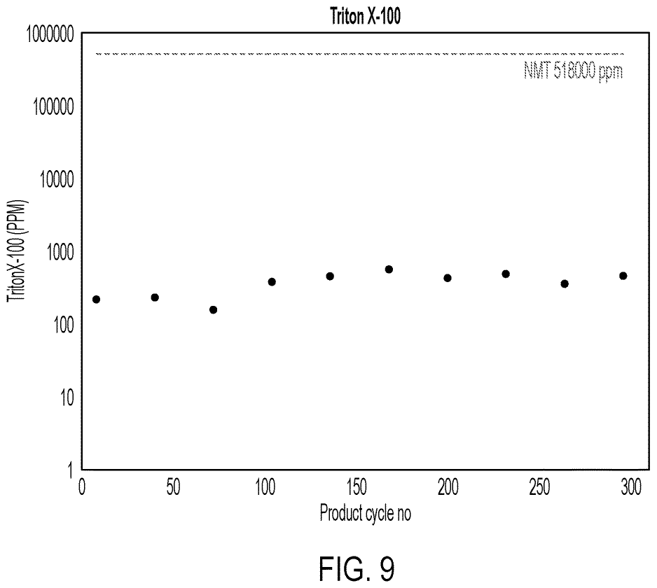

FIG. 9 shows that protein A mainstream rTX-100 concentration (ppm) remained consistent throughout the duration of the first column pack. Levels of rTX-100 increased approximately 3-fold after the column was re-packed but remained consistent for the remainder of the study meeting the acceptance criteria of ≤518,000 ppm with values between 371 and 572 ppm (post-repack). The level of rTX-100 is displayed on a logarithmic scale in FIG. 9 due to the offset between the acceptance criteria and recorded values. This confirms the capability of the resin to reduce rTX-100 across 304 product cycles.

Residual Insulin (rInsulin)

The protein A mainstream rInsulin concentration (μU/ml) remained consistent throughout the duration of this study meeting the acceptance criteria of ≤20 μU/ml (no data shown). This confirms the ability of the resin to reduce process related impurities to acceptable levels across 304 product cycles.

### Protein A Lifetime Study Comparison

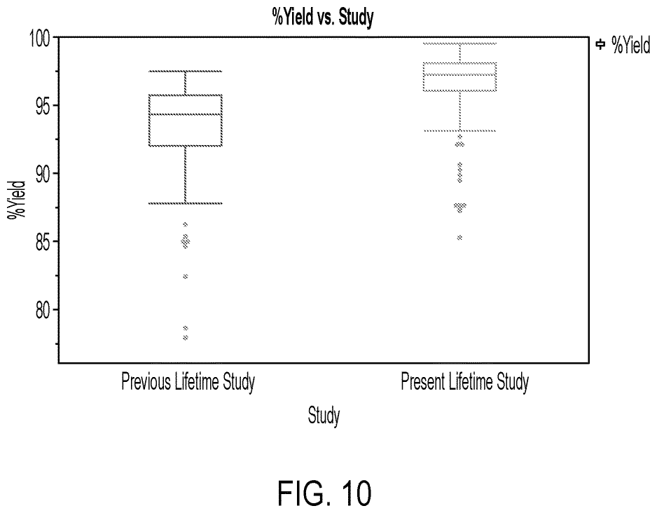

The present study demonstrated higher and more consistent yields than a previous dulaglutide MSS LX protein A chromatography lifetime study, which included a cleaning step of a static hold with a solution containing 1% acetic acid, 1% phosphoric acid followed by a sanitization wash with 50 mM NaOH and 1 M NaCl (FIG. 10, note that the gray dots in FIG. 10 are outliers). The final yield achieved in this study (97.8%, 304 cycles) was significantly increased compared to the previous protein A chromatography lifetime study (88.9%, 112 cycles). Thus, these results demonstrate an improved method for cleaning a protein A chromatography matrix that extends the usable lifetime of the protein A chromatography matrix. This introduction of the additional 0.01 M NaOH caustic wash in the cleaning protocol described in this study nullified the increases in pre-peak observed in the previous lifetime study.

### CONCLUSIONS

This study demonstrated a consistently high yield across 304 runs while maintaining a dynamic binding capacity higher than the target load until the end of the resin lifetime, indicating that the additional caustic exposure should not impact the % yield CIPC of ≥70% across the lifetime of the resin. No negative trends were observed for residual host cell protein, residual DNA, residual insulin, residual protein A and residual Triton X-100 tested across 304 dulaglutide purification cycles, where the additional step of washing the protein A chromatography matrix with a caustic solution containing 0.01 M NaOH had no impact on the residual clearance. Quality attributes that indicate stability, as measured by SEC, were largely unchanged over the 304 product cycles performed.

This study demonstrates that a protein A chromatography matrix for the processing of dulaglutide is capable of purifying dulaglutide with acceptable product quality and with acceptable unit operation performance for at least 304 purification cycles by using the improved cleaning protocol that includes an additional caustic wash step (0.01 M NaOH). This number of cycles could be extended even beyond 304 cycles because, surprisingly, none of the evaluation criteria assessed in the study described above were negatively impacted through 304 cycles.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method for cleaning a protein A chromatography matrix that has previously been used to purify dulaglutide, the method comprising sequentially washing a chromatography column comprising the protein A chromatography matrix with:
a) a first solution comprising one or more acid;
b) a second solution comprising Tris; and
c) a third solution comprising 0.001-0.075 M NaOH.

2. The method of claim 1, wherein the first solution comprises acetic acid.

3. The method of claim 1, wherein the first solution comprises phosphoric acid.

4. The method of claim 1, wherein the first solution comprises acetic acid and phosphoric acid.

5. The method of claim 1, wherein the first solution comprises 0.5-5% acetic acid.

6. The method of claim 1, wherein the first solution comprises 0.5-1.5% acetic acid.

7. The method of claim 1, wherein the first solution comprises about 1% acetic acid.

8. The method of claim 1, wherein the first solution comprises 0.5-5% phosphoric acid.

9. The method of claim 1, wherein the first solution comprises 0.5-1.5% phosphoric acid.

10. The method of claim 1, wherein the first solution comprises about 1% phosphoric acid.

11. The method of claim 1, wherein the first solution comprises about 1% acetic acid and about 1% phosphoric acid.

12. The method of claim 1, wherein the second solution has a pH of 7-9.

13. The method of claim 1, wherein the second solution has a pH of about 8.

14. The method of claim 1, wherein the second solution comprises 10-100 mM Tris.

15. The method of claim 1, wherein the second solution comprises 25-75 mM Tris.

16. The method of claim 1, wherein the second solution comprises about 50 mM Tris.

17. The method of claim 1, wherein the third solution comprises 0.005-0.05 M NaOH.

18. The method of claim 1, wherein the third solution comprises 0.009-0.015 M NaOH.

19. The method of claim 1, wherein the third solution comprises about 0.01 M NaOH.

20. The method of claim 1, wherein the protein A chromatography matrix is washed with 1-10 column volumes of the first solution.

21. The method of claim 1, wherein the protein A chromatography matrix is washed with 2-3 column volumes of the first solution.

22. The method of claim 1, wherein the protein A chromatography matrix is washed with 2-10 column volumes of the second solution.

23. The method of claim 1, wherein the protein A chromatography matrix is washed with 1-2 column volumes of the second solution.

24. The method of claim 1, wherein the protein A chromatography matrix is washed with 2-10 column volumes of the third solution.

25. The method of claim 1, wherein the protein A chromatography matrix is washed with 2-3 column volumes of the third solution.

26. The method of claim 1, wherein the protein A chromatography matrix is washed with the first solution at a flow rate of about 270 cm/hr.

27. The method of claim 1, wherein the protein A chromatography matrix is washed with the second solution at a flow rate of about 270 cm/hr.

28. The method of claim 1, wherein the protein A chromatography matrix is washed with the third solution at a flow rate of about 270 cm/hr.

29. The method of claim 1, wherein the chromatography column has a diameter of 75-150 cm.

30. The method of claim 1, wherein the chromatography column has a diameter of about 100 cm.

31. The method of claim 1, wherein the protein A chromatography matrix has an average particle size of 80-90 μm.

32. The method of claim 1, wherein the protein A chromatography matrix has an average particle size of about 85 μm.

33. The method of claim 1, wherein the protein A chromatography matrix a comprises a protein A ligand that has increased stability under alkali conditions relative to wild type Staphylococcus aureus protein A.

34. The method of claim 1, wherein
a) the first solution comprises about 1% acetic acid and about 1% phosphoric acid;
b) the second solution comprises about 50 mM Tris at a pH of about 8; and
c) the third solution comprises about 0.01 M NaOH.

35. The method of claim 34, wherein the protein A chromatography matrix is washed with:
a) 2-3 column volumes of the first solution;
b) 1-2 column volumes of the second solution; and
c) 2-3 column volumes of the third solution.

36. The method of claim 35, wherein the protein A chromatography matrix is washed with the first, second, and third solutions at a flow rate of about 270 cm/hr.

37. The method of claim 36, further comprising washing the protein A chromatography matrix with about 2 column volumes of the second solution after the protein A chromatography matrix is washed with the third solution.

38. The method of claim 37, further comprising washing the protein A chromatography matrix with a fourth solution comprising about 0.1 M NaOH.

39. The method of claim 38, wherein the protein A chromatography matrix is washed with about 2 column volumes of the fourth solution.

40. The method of claim 39, wherein the protein A chromatography matrix is washed with the fourth solution at a flow rate of 100-140 cm/hr.

41. The method of claim 40, wherein the protein A chromatography matrix is washed with the fourth solution for about 25 minutes.

42. The method of claim 1, wherein the protein A chromatography matrix is washed with the first solution, second solution, and/or third solution in an upflow or downflow direction.

43. The method of claim 1, wherein the protein A chromatography matrix has less than 1% carryover from the previous use after the protein A chromatography matrix is cleaned.

44. The method of claim 1, wherein the protein A chromatography matrix has less than 0.1% carryover from the previous use after the protein A chromatography matrix is cleaned.

45. The method of claim 1, wherein the protein A chromatography matrix is used for 200-500 cycles of dulaglutide purification.

46. The method of claim 1, wherein the protein A chromatography matrix is used for 300-400 cycles of dulaglutide purification.

47. The method of claim 1, wherein the protein A chromatography matrix is used for about 304 cycles of dulaglutide purification.

48. The method of claim 1, wherein the method does not include a static hold of the first solution.

49. The method of claim 1, wherein the method does not include a static hold of the second solution.

50. The method of claim 1, wherein the method does not include a static hold of the third solution.

51. Dulaglutide produced by the method of claim 1.

52. A composition comprising dulaglutide produced by the method of claim 1.


SEQUENCE LISTING (4 sequences)

SEQ ID NO: 1 - protein, 31 aa, Homo sapiens
HAEGTFTSDVSSYLEGQAAKEFIAWLVKGRG

SEQ ID NO: 2 - protein, 31 aa, synthetic construct
HGEGTFTSDVSSYLEEQAAKEFIAWLVKGGG

SEQ ID NO: 3 - protein, 5 aa, synthetic construct
GGGGS

SEQ ID NO: 4 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG